(19) United States
(12) Patent Application Publication
(10) Pub. No.: US 2025/0359805 A1
(43) Pub. Date: Nov. 27, 2025

(54) SYSTEMS AND METHODS FOR IMPROVED NEURODATA CAPTURE

(71) Applicant: Nuream, Inc., Wilmington, NC (US)

(72) Inventors: Robert S. COOLEY, JR., Wilmington, NC (US); Nathan MUNTON, Wilmington, NC (US); Lauren MUNTON, Wilmington, NC (US); David ZAR, Wilmington, NC (US); Lloyd SMITH, Wilmington, NC (US)

(21) Appl. No.: 19/215,785

(22) Filed: May 22, 2025

Related U.S. Application Data
(60) Provisional application No. 63/650,568, filed on May 22, 2024.

(51) Int. Cl. A61B 5/369; A61B 5/00

(52) U.S. Cl. CPC A61B 5/369; A61B 5/0006; A61B 5/6804; A61B 2562/046; A61B 2562/066

(57) ABSTRACT

A system and method for improved capture of neurodata and other biometric information includes an array of sensing positions integrated into a textile and configured to collect signals associated with a subject and a processor in communication with the array of sensing positions, where the processor is configured to identify biometric information within the signals. The array of sensing positions being distributed over an area of the textile to provide a “swarm sensing” capability to collect brain waves (or other related data) without discrete sensors on the head.

### CROSS-REFERENCE TO RELATED APPLICATIONS

This is a non-provisional of, and claims the benefit of the filing date of, U.S. provisional patent application No. 63/650,568, filed May 22, 2024, entitled “SYSTEMS AND METHODS FOR IMPROVED NEURODATA CAPTURE,” the entirety of which application is incorporated by reference herein.

### FIELD OF THE DISCLOSURE

The subject matter disclosed herein relates generally to systems and method for collecting and analyzing electrical stimuli derived from a human subject. More particularly, the subject matter disclosed herein relates to sensors that are configured to capture neurodata and other biometric data.

### BACKGROUND OF THE DISCLOSURE

The brain moves through many sleep stages throughout the night, and a person's brain wave patterns can provide a clear indication on how well the person sleeps. For example, measurements of defined frequency ranges, changes to frequency, defined amplitude ranges, and changes to amplitude have been associated with different sleep stages. Accurately tracking these sleep stages can provide a general guide to the quality of sleep. In addition, knowing how a person sleeps, over time, can be a reliable indicator to future health, wellness, productivity, performance, and disease state. In this regard, because knowing a person's sleep variables and how they affect the brain at night relates directly to sleep quality, there is a desire to be able to collect and analyze brain wave data to identify issues.

Collecting brain data is not new, with electroencephalogram (EEG) data being the conventional method for collecting the data. To collect data in this way, however, subjects typically must wear headbands or a cap with discrete sensors positioned at selected locations about the device, which can often be invasive, uncomfortable, and disrupting to daily activity. In other words, the devices currently used to collect brain data are not usable day-to-day. As a result, because present data capture technologies tend to be invasive and uncomfortable, it is currently difficult to collect usable data, which further limits the predictive capability of present neurodata collection. Accordingly, neurodata is not widely used in performance, wellness, and clinical applications.

It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A system and method for improved capture of neurodata and other biometric information is disclosed. In some examples, the system for collecting biometric information includes an array of sensing positions integrated into and/or formed in a textile and configured to collect signals associated with a subject, the array of sensing positions being distributed over an area of the textile and a processor in communication with the array of sensing positions, wherein the processor is configured to identify biometric information within the signals.

A method for collecting biometric information is also disclosed. The method can include positioning a textile against a portion of a subject's body, wherein the textile comprises an array of sensing positions integrated therein, collecting signals from the array of sensing positions, isolating biological signals associated with the subject from the signals collected from the array of sensing positions, and analyzing the biological signals to identify biometric information of the subject. In some examples, the method is implemented by a non-transitory computer-readable storage medium having executable instructions stored thereon.

In any preceding or subsequent example, the array of sensing positions can include a plurality of discrete sensors that are each attached to the textile. Alternatively, the array of sensing positions can include a plurality of conductive fibers integrated into the textile and/or conductive inks imprinted on the textile.

In any preceding or subsequent example, the textile can be a pillowcase, where the array of sensing positions is distributed over a majority of an area of the pillowcase. Other implementations can include the textile being used in clothing, sleeves for arms or legs, wearable wristband materials, vehicle seats, materials in mattresses, or any of a variety of similar applications.

In any preceding or subsequent example, the processor can be configured for continuously collecting signals from all elements in the array of sensing positions and identifying a subset of the array of sensing positions that are determined to be in contact with the subject. In such examples, the processor can further be configured to identify the biometric information within the signals by analyzing only the signals collected from the subset of the array of sensing positions.

In any preceding or subsequent example, a data collection module can be connected to the array of sensing positions, where the data collection module is configured to transmit the signals collected by the array of sensing positions to the processor. In some examples, an embroidered bus can be integrated into the textile and configured to electrically connect the array of sensing positions to the data collection module. In some examples, one or more of the array of sensing positions is configured to operate as a reference electrode. In this configuration, the data collection module can be configured to provide a bias signal to the reference electrode.

In any preceding or subsequent example, isolating biological signals associated with the subject can include recognizing patterns in the signals that correspond to expected biometric data patterns. In some examples, recognizing patterns in the signals can include using an artificial intelligence or machine learning model to identify neurodata from within the signals. In some such examples, analyzing the neurodata can include comparing the neurodata identified from within the signals to historical neurodata of the subject that is used as training data for the artificial intelligence or machine learning model.

In any preceding or subsequent example, isolating neurodata associated with the subject can include prioritizing sensing positions from among the array of sensing positions that are determined to be closest to a brain of the subject.

In any preceding or subsequent example, the system and method can further be used to identify additional biometric indicators within the signals collected from the array of sensing positions. For example, the additional biometric indicators can include but are not limited to heart rate, heart rate variability (HRV), blood oxygen level (SpO2), temperature, and respiration rate.

Examples of the present disclosure provide numerous advantages. For example, the present systems and methods can collect brain waves (or other related data) without discrete sensors on the head. Such systems and methods can thus be less invasive and more comfortable than current monitoring modalities.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates a schematic representation of a system for collecting biometric information in accordance with one or more features of the present disclosure;

FIG. 2 is a plan view of a textile including an array of sensing positions configured for collecting biometric information in accordance with one or more features of the present disclosure.

FIG. 3 is a plan view of a textile including an array of sensing positions configured for collecting biometric information in accordance with one or more further features of the present disclosure.

FIG. 4 illustrates a perspective side view of a system for collecting biometric information being used by a subject in accordance with one or more features of the present disclosure.

FIG. 5 is a process flow chart including steps in a method for collecting biometric information in accordance with one or more features of the present disclosure.

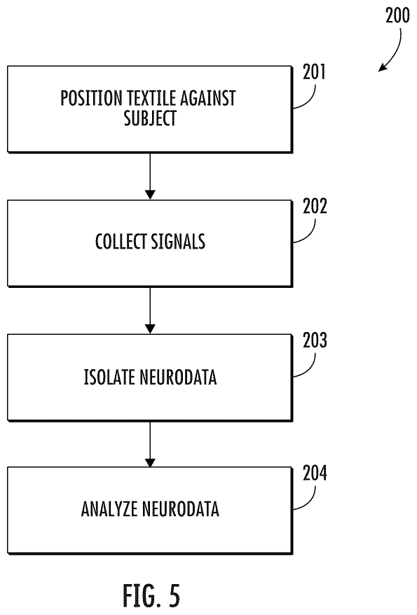

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a system and method for improved capture of neurodata and other biometric information will now be described more fully herein with reference to the accompanying drawings, in which one or more features of the system and method will be shown and described. It should be appreciated that the various features may be used independently of, or in combination, with each other. It will be appreciated that the system and method as disclosed herein may be embodied in many different forms and may selectively include one or more concepts, features, or functions described herein. As such, the system and method should not be construed as being limited to the specific examples set forth herein. Rather, these examples are provided so that this disclosure will convey certain features of the system and method to those skilled in the art.

In accordance with one or more features of the present disclosure, systems and methods for improved capture of electrical stimuli, including neurodata and other biometric indicators is disclosed. In one aspect, the present subject matter provides a non-invasive, multi-modal brain wave capture system. In some examples, biological signals can be captured using a fabric or other textile from which sensing data can be directly derived. As used herein, the term “textile” is intended to describe materials including but not limited to woven or non-woven fabrics or substrates including an arrangement of fibers, yarns, filaments, and/or threads.

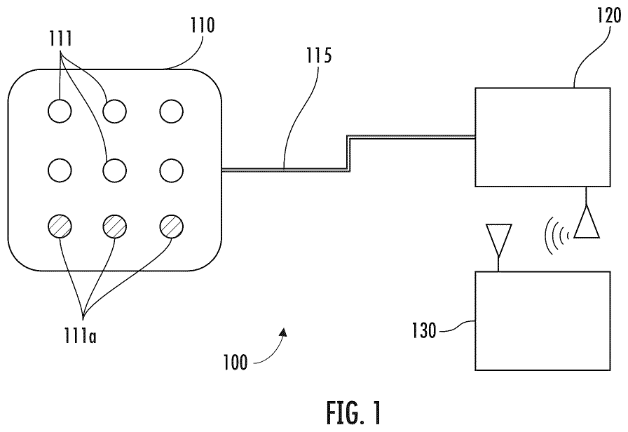

Referring to an embodiment illustrated in FIG. 1, in some examples, the system for collecting biometric information, generally designated 100, in accordance with one or more features of the present disclosure is shown. As illustrated, the system 100 can include an array of sensing positions 111 integrated into a textile 110 that can be positioned against a subject's body. In particular, the array of sensing positions 111 can be distributed over an area of the textile 110. In some examples, the textile 110 can be provided in the form of a pillow covering and/or pillowcase for sleep monitoring, with the array of sensing positions 111 being distributed over a majority of the surface area of the textile 110. In particular, in some examples, the array of sensing positions 111 can be distributed over an area greater than 75% of the total surface area of the textile 110. In such a configuration, the array of sensing positions 111 can be used to collect the brain wave and/or other electrical stimuli. In some examples, neurodata can be collected in addition to any of a variety of other biometric indicators (e.g., electrical signals from the heart, brain, muscles, or nerves) that can more particularly characterize an individual subject's physical health, performance, wellness, mental health, and/or mental wellbeing. For example, the collected biometric data can further be correlated to gold-standard EEG information to categorize sleep cycles.

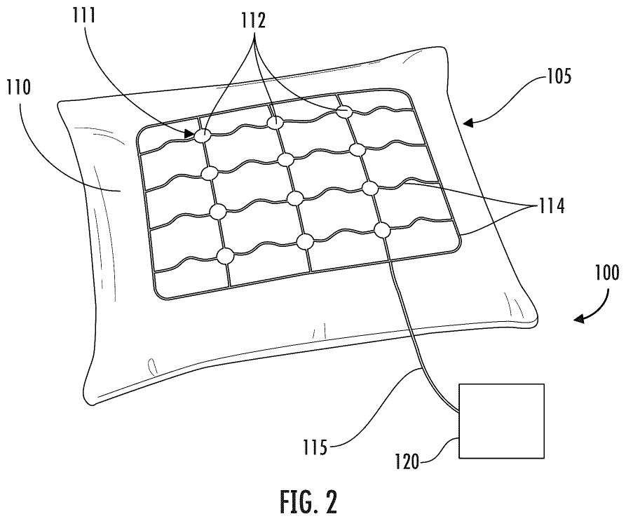

Referring to one example implementation of the system 100 illustrated in FIG. 2, the textile 110 can be provided as a pillowcase that is positioned over a pillow 105 and into which a plurality of discrete electrodes 112 are integrated to serve as the array of sensing positions 111. In some examples, each of the electrodes 112 is individually stitched or otherwise adhered to the textile 110 in a selected position within a predetermined array. In the illustrated embodiment, only twelve electrodes 112 are shown, but those having ordinary skill in the art will recognize that any number of electrodes 112 can be included to provide a desired area coverage over the textile 110. For instance, some configurations can include at least 32 electrodes 112 (e.g., arranged in an 8×4 grid) distributed over an area of the textile 110. Those having ordinary skill in the art will recognize, however, that the electrodes 112 can be arranged in any other number or arrangement depending on the desired configuration of the system 100. The system 100 can further include conductive yarns or other flexible connectors that are connected to each of the electrodes 112 and serve as an embroidered bus 114 that is configured to provide electrical communication to each of the electrodes 112.

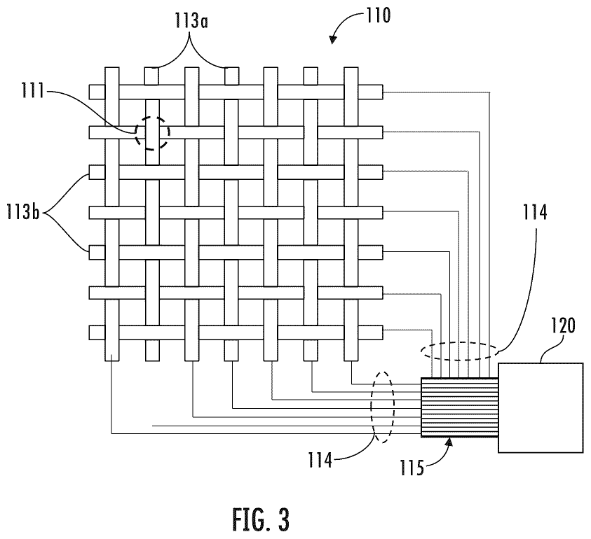

Alternatively, referring to a further example implementation of the system 100 illustrated in FIG. 3, the textile 110 can be at least partially composed of conductive fibers 113a/113b that are integrated into the textile 110. In some examples, such conductive fibers 113a/113b can include any of a variety of materials, including but not limited to spun copper, silver, gold, or aluminum; metal alloys such as nichrome or brass; nanotechnologies; conductive polymers such as poly(2,3-dihydrothieno-1,4-dioxin)-poly(styrenesulfonate) (PEDOT: PSS) or polyaniline (PANI); gel electrolytes; semiconductors such as silicon or gallium arsenide (GaAs); or carbon-based materials such as graphite, graphene materials, or carbon nanotubes. In some examples, the conductive fibers 113a/113b can be provided in a woven format, such as is illustrated in FIG. 3, in which the conductive fibers 113a/113b are arranged as warp fibers 113a and weft fibers 113b that cross each other substantially at right angles. In this arrangement, the intersections of the warp fibers 113a and the weft fibers 113b can serve as the array of sensing positions 111. In such a configuration, rather than an array of discrete sensors, the portion of the textile 110 (e.g., the entire fabric or a certain percentage of fibers in the overall woven and/or non-woven material) having the conductive fibers 113a/113b integrated therein is able to collect the brain wave stimuli as a distributed fabric-as-a-sensor (FaaS) array.

Although the conductive fibers 113a/113b are shown in FIG. 3 as being arranged in a plain weave configuration, those having ordinary skill in the art will recognize that other woven configurations can similarly be used to define the array of sensing positions 111 among the conductive fibers 113a/113b. Alternatively, the conductive fibers 113a/113b can be integrated into the textile 110 in any of a variety of other textile formats, including but not limited to knitted or non-woven fabrics. In any configuration, the conductive fibers 113a/113b can comprise the entirety of the fibers used to create the textile 110 or they can be interspersed with more conventional fibers. Those having ordinary skill in the art will recognize that the specific density of conductive fibers 113a/113b per unit area of the textile 110 can vary based on the materials used and the desired density of sensing positions 111. In some examples, the density of the conductive fibers 113a/113b is sufficiently high that inter-sensor distance between the array of sensing positions 111 is within a resolution limit, allowing the entire surface of the textile 110 to operate as a substantially continuous sensing area. In some further alternative examples, the textile 110 can be composed of conventional materials but can be imprinted with conductive inks that define the array of sensing positions 111 and associated electrical leads and connectors.

In any configuration, distributing the array of sensing positions 111 over an area of the textile 110 allows for multiple touch positions on the head and/or neck of the subject when in contact with the pillowcase during sleep. In addition, the number and distribution of the array of sensing positions 111 need not be specifically arranged to align with any predetermined sensing positions on the subject's body. Rather, the array of sensing positions 111 can be distributed over a larger area of the textile 110 than is needed to provide sensing of the subject such that, if and when the subject moves during sleep, a different subset of the array of sensing positions 111 can still be in contact with the subject. In some examples, the array of sensing positions 111 can be distributed over an area of the textile 110 that is at least about 20 inches by about 20 inches. In such a configuration, where a standard pillowcase is about 20 inches by about 26 inches, a majority of the pillow's surface can be configured to operate as an active sensing area. As a result, so no matter where the subject's head is during sleep, at least a minimum number of the array of sensing positions 111 can contact the head as necessary for signal detection.

In some examples, one or more of the sensing positions 111 can be configured to operate as a reference and/or bias electrode 111a that is arranged to be in continuous contact with the subject. In particular, in some examples, a linear set of reference electrodes 111a can be provided along a bottom of the textile 110 of the pillowcase. In this arrangement, at least one of the reference electrodes 111a can be in contact with the subject's neck, which can in many cases provide an increased likelihood of creating high-fidelity contact with the subject due to less interference from hair. The remaining locations of the array of sensing positions 111 can then be positioned away from this ground/reference set, giving a larger signal on them. In some examples, the one or more reference electrodes 111a can be provided with a bias signal (e.g., driven right leg (DRL)) to identify and enable suppression of common-mode interference.

Regardless of the particular configuration, the array of sensing positions 111 can be configured to collect signals associated with the subject. For this data collection, the system 100 can further include a processor 130 in communication with the array of sensing positions 111 that is configured to identify the biometric information within the signals. As used herein, the term “processor” can be used to describe a processor, processing circuit, and/or microcontroller that is directly connected to the textile 110, connected wirelessly, or located remotely from the textile 110. In some examples, the system 100 can be configured to wirelessly send collection data from the textile 110, move data for further computation in a cloud-based or other remote system, and return results to on the subject's personal phone/device.

As indicated above, the array of sensing positions 111 can be distributed over an area of the textile 110 that is sufficiently large such that a subset 111′ of the sensing positions 111 will be in contact with or are in sufficiently close proximity to the subject such that the signals collected by the subset 111′ of the sensing positions 111 can be analyzed together to identify biometric information within the signals. In some examples, collected data can be analyzed to identify the position of the data collection relative to the subject's body (e.g., in contact with a portion of the subject's head), and this identification can be reevaluated regularly to account for movement relative to the array of sensing positions 111. In this way, the configuration and operation of the system 100 can account for movement and/or muscle artifacts during sleep or any of a variety of environmental noises (e.g., other people, pets, televisions or other electronic devices).

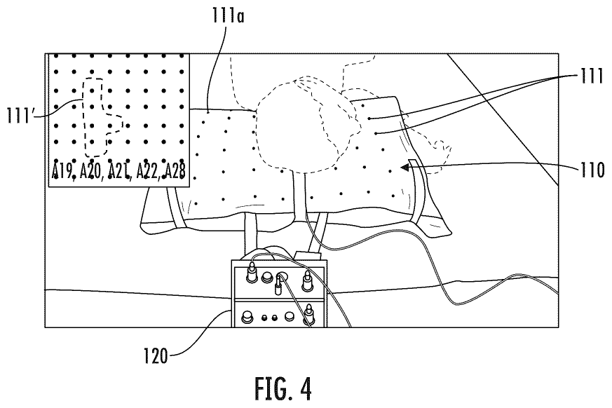

In some examples, signals from all of the sensing positions 111 can be collected continuously, and analysis of the outputs of each of the sensing positions 111 can identify which of the sensing positions 111 define the subset 111′ that is in sufficiently close proximity to the subject to collect signal data at a given time within the data collection period, such as is shown in an example configuration illustrated in FIG. 4. In this way, the present system 100 can provide a “swarm sensing” capability to collect brain waves (or other related data) without discrete sensors on the head. Such a system can be less invasive and more comfortable than current monitoring modalities. Those having ordinary skill in the art will recognize that this swarm sensing capability can be achieved regardless of the particular configuration of the textile 110. As indicated above, the sensing positions 111 can be provided across the textile 110 in the form of discrete electrodes 112, an array of conductive fibers 113a/113b, or in an of a variety of other sensor configurations suitable for collecting the biological signals disclosed herein.

In some examples, the processor 130 can be integrated into the textile 110 to provide a standalone system. Alternatively, in some examples, the processor 130 can be independent from the textile 110, and the system 100 can include a data communication arrangement configured to collect the signals from the array of sensing positions 111 and transmit them to the processor 130. In this regard, in some examples, the system 100 can include a data collection module 120 that is connected to the array of sensing positions 111. The data collection module 120 can include appropriate front-end electronics that are configured to receive and store the signal data collected from the subject by the array of sensing positions 111. Such front-end electronics can include multiplexers and signal conditioners that are configured to ensure clean, selectable inputs are provided to the processor 130. In some examples, the data collection module 120 can be a dedicated electronic device that is designed and configured particularly for this purpose. Alternatively, in some other examples, the data collection module 120 can be implemented in software executed by a processor or processing unit of a general-purpose computer or personal electronic device (e.g., a cell phone).

In some examples, the system 100 includes a connection cable 115 that is connected to each of the array of sensing positions 111 and the data collection module 120. In the configuration shown in FIG. 2, for example, the connection cable 115 can be connected to each of the electrodes 112 by the embroidered bus 114. Similarly, in the configuration shown in FIG. 3, the conductive fibers 113a and 113b can be connected to the connection cable 115 by the embroidered bus 114. In these examples, the connection cable 115 is configured to provide a robust electrical/mechanical connection between the textile 110 and the data collection module 120. Alternatively, in some examples, the connection cable 115 can be considered optional, where the data collection module 120 can be connected directly to the embroidered bus 114.

In any configuration, in some examples, the data collection module 120 can be arranged at or near the textile 110 at a position such that the data collection module 120 and/or the connection cable 115 will not interfere with regular use of the pillow 105 to which the textile 110 is associated. In some examples, the data collection module 120 can be sewn into a pocket under the pillow 105. In some examples, the data collection module 120 can be battery-powered such that the textile 110 need not be positioned near a dedicated power source, such as a wall plug. In some such examples, the batter power of the data collection module 120 can be rechargeable without removing it from the system, such as via a common USB cable (e.g., a 15 W-5V/3A USB-C cable). In such a configuration, it can be advantageous for the battery power of the data collection module 120 to be sized to provide at least 8 hours of continuous operation on a full charge to allow the collection of data over a typical sleep duration.

Likewise, the data collection module 120 can be configured to store at least 8 hours of sleep data. In this regard, in some examples, the data collection module 120 can include a non-volatile internal memory, a removable SD card, or any of a variety of other data storage media that has a sufficient data capacity to store at least 8 hours of signal data.

In this configuration, because the data collection module 120 can be configured primarily to receive and store the signal data collected from the subject by the array of sensing positions 111, it need not include all of the functionality necessary to analyze the signals. Instead, the data collection module 120 can transmit the signal data to the processor 130 for such analysis. In some examples, the data collection module 120 can be wirelessly connected to the processor 130 such that the processor 130 need not be physically positioned at or near the textile 110. In some examples, the processor 130 can be connected to the data collection module 120 using any of a variety of wireless data transfer protocols, including but not limited to Bluetooth, Wi-Fi, or other secure wireless communication standards.

From the signals received from the data collection module 120, in some examples, the processor 130 can be configured to correlate the received signals to the positions on the subject's body where those signals are most likely to be measurable in a manner substantially analogous to conventional EEG collection systems in which discrete sensors are glued/attached to the subject's head. For this purpose, the data collection module 120 and/or the processor 130 can be configured for receiving and analyzing signals having characteristics that are generally associated with the types of biometric data to be collected. In some examples, these components can be configured to receive and analyze signals having signal amplitudes ranging from about 20 to about 100 μV and from about 0.5 mV to about 5 mV (ECG range). These signal ranges can be on top of any electrode offset values. The components can further be configured to be operable in bandwidths between about 0.05 Hz to about 100 Hz (e.g., to capture ECG data). In some examples, the components can exhibit sampling rates up to about 4 kHz, with a resolution of greater than about 18 bits (e.g., 24 bits), RMS noise less than about 1 μV, and very high input impedance (e.g., greater than about 300 MΩ).

Regardless of the particular configuration of the components of the non-invasive, multi-modal brain wave capture system used, in another aspect, the present subject matter provides a method 200 for collecting biometric information. In some examples, the method 200 includes a positioning step 201 in which the textile 100 against a portion of a subject's body. A collection process 202 can then include collecting signals from the array of sensing positions 111 integrated in the textile 110. As discussed above, in some examples, the collection process 202 can include continuously receiving signals from all of the sensing positions 111 and analyzing the signals from each of the sensing positions 111 to identify which of the sensing positions 111 define the subset 111′ that are determined to be in contact with the subject so as to provide “good” signals from which relevant data can be extracted. For example, the collection process 202 can include comparing the signals of each of the sensing positions 111 against one or more reference and/or bias signal (e.g., from one or more reference electrodes 111a) to distinguish valid signals from common-mode interference. Alternatively or in addition, in some examples, the collection process 202 can include correlating the signals of each of the sensing positions 111 to gold-standard EEG sleep stage data.

The method can further include an isolation process 203 in which the desired biological signals are identified from within the collected signals (or subset of signals). Because neurodata is generally at a much lower amplitude than other biometric data (e.g., ECG, motor data), the isolation process 203 can involve isolating neurodata from other received signals based on recognition of the patterns in the signals and/or an identification of sensing positions that are closest to the brain such that confounding signals are less dominant. In some examples, the identification includes filtering noise from the signals to isolate the neurodata. In some examples, the processor 130 can include an artificial intelligence and/or machine learning model that is configured to identify the neurodata from within the collected signals.

The method can further include an analyzing process 204 in which the biological signals isolated from the signals is analyzed to characterize the subject's sleep. In some examples, the analyzing process 204 can be performed using one or more algorithms. For example, one or more algorithms may be used to monitor brain activity during sleep and create a data dashboard displaying brain activity insights. It is now widely accepted that there are four sleep stages that a person transitions through during a sleep session: N1, N2, N3, and REM. N1 is characterized as a short period of light sleep that lasts for around 1-5 minutes in which a person's heart rate, breathing, eye movements, and brain waves slow down. The muscles also relax, although they may twitch occasionally. N2 is a period of deeper sleep in which the muscles relax further, eye movements stop, and body temperature drops. During the first sleep cycle of the night, this N2 stage can last for around 25 minutes, lengthening with each new sleep cycle. N3 is the deepest stage of sleep and the hardest to awaken from. Heart rate, breathing, and brain waves become regular during this stage, and a person will experience the deepest sleep during the first half of the night. Finally, the last stage of the sleep cycle is REM sleep. During this stage, the eyes move quickly and rapidly from side to side, and breathing quickens and becomes more erratic. A person typically moves cyclically through these stages throughout the sleep session, with four to five cycles typically occurring in a sleep session.

In some examples, the analyzing process 204 can identify which sleep stage a person is experiencing at a given time based on analysis of the wave type, frequency, and/or amplitude of the brain wave activity measured by the system 100. Commonly, the N1 sleep stage is characterized by alpha waves having frequencies in a range of between about 8 and about 13 Hz and/or beta waves having frequencies in a range of between about 13 and about 30 Hz, with amplitudes of about 30 μV; the N2 sleep stage is characterized by alpha waves having frequencies in a range of between about 8 and about 13 Hz and/or theta waves having frequencies in a range of between about 4 and about 7 Hz, with amplitudes in a range between about 20 μV and about 100 μV; the N3 sleep stage is characterized by delta waves having frequencies in a range of between about 0.5 and about 4 Hz, with amplitudes in a range between about 20 μV and about 100 μV; and the REM sleep stage is characterized by alpha waves having frequencies in a range of between about 8 and about 13 Hz and/or beta waves having frequencies in a range of between about 13 and about 30 Hz, with amplitudes of about 30 μV. Transitions between stages are typically not abrupt, and thus the analyzing process 204 can use signal processing and/or artificial intelligence (AI) models to delineate between sleep stages in the data.

Alternatively or in addition, the analyzing process 204 can further include analyzing additional biometric indicators, including but not limited to heart rate, heart rate variability (HRV), blood oxygen level (SpO2), temperature, respiration, and/or body movement data. In some examples, these biometric indicators can be used for comparison with the neurodata to provide a comprehensive assessment of the subject's activity. In addition, in some examples, the collected biometric indicators can also be used to infer other biosignals from the body.

Further applications can include algorithms for early detection of neurological episodes, such as seizures. Additional algorithms include detection and/or treatment of any of a variety of other conditions and/or disease states, including but not limited to diet problems, substance abuse, mental health issues, multiple sclerosis (MS), post-traumatic stress disorder (PTSD), epilepsy, Alzheimer's, Parkinson's, schizophrenia, or dementia. For these purposes, the present systems and methods may be able to provide early warning of neurological issues, optimization of day-to-day performance (or in high pressure situations), fatigue monitoring, surgical planning, and/or recovery monitoring.

As discussed above, in some examples, the algorithms used to analyze the collected biological signals include artificial intelligence and/or machine learning-based algorithms. At least a portion of the desired analysis can be conducted locally, with a phone or other personal electronic device acting as the processor 130 in communication with the textile 110. Alternatively, the collected biological signals can be transmitted to a remote and/or cloud-based system configured to operate as the processor 130 such that the processing can be offloaded from the local collection device. Even in such remote processing configurations, however, the local device can be used as the data collection module 120 while also providing for some preliminary processing, which can include but not be limited to data cleaning, amplification, and/or computation as close to the individual as the technology allows.

In some examples, the systems and methods disclosed herein can account for individual variation in the neurodata and other biometric indicators collected and analyzed. It is recognized that each human's brain operates at different portions but generally within the accepted ranges (e.g., some higher than average and some lower). To account for this variability, the present systems and methods can use an individual's brain wave information to train the applied algorithms to model the characteristics of the individual subject and provide an individual solution rather than merely comparing collected data to a standard model.

In some examples, this individual characterization can allow the individual to run their own in bedroom sleep studies and provide predictive opportunities for improvement. In such a “closed loop” configuration, the subject can essentially run these sleep studies to see if changes to sleep environment, sleep preparation, sleep hygiene, and/or any protocols such as sleep apnea devices, pharma/biotech, alcohol, over the counter sleep aids, or the like can be correlated to any sustained changes to more restorative sleep. This trending and information can be provided to the individual in a “human in the loop” (the consumer) sleep score and/or decision support dashboards.

Alternatively or in addition, in some examples, the collected data can be shared among a small group, such as a group of family members, caregivers, medical practitioners, and/or other accountability partners. In some examples, protocols for this kind of “extended closed loop” data exchange can be similar to standard electronic medical record (EMR)/electronic health record (HER) systems used by clinicians, family members, care givers, EMT emergency, large corporate wellness providers to industry, performance trackers, and/or leadership in the military. In yet a further alternative, in some examples, the collected data can be aggregated together to identify population-level trends, which can be used as inputs for industry research and development, topics for academic research, areas of focus for clinical support, and the like. In some examples, this kinds of “meta closed-loop” can include steps of removing any individually-identifying information from the collected data, storing the anonymized aggregated data at a meta level, and cataloguing for future use and sale to many potential users.

In any implementation, the present systems and methods can be configured to maintain neurodata security at collection, during transmission, during dashboard preparation, and at eventual annotated (anonymous) storage and usage.

While specific features and/or examples have been shown and described, it is envisioned that modifications can be made. For example, although the present system 100 is shown and described herein in the context of this pillow covering and/or pillowcase, those having ordinary skill in the art will recognize that the textile 110 into which the array of sensing positions 111 is integrated can similarly be applied to any of a variety of further applications, including but not limited to T-shirts for daily tracking, assessment, and delivery of other biometric data, or in other wearable devices such as wrist bands or watches, sleeves for arms or legs, vehicle seats (e.g., in automobiles, trains, planes, trucks), materials in mattresses, or any of a variety of similar applications. Further, in addition to the sleep and general health tracking that may be supported by the present systems and methods, the principles disclosed herein can also be applied to improved gaming and augmented reality (AR), virtual reality (VR), mixed reality (MR), and/or extended reality (XR) training through advanced neurosensing scenarios. In addition, the present systems and methods can be adapted to applications for identifying and monitoring substance abuse/overuse.

The subject matter disclosed herein can be implemented in or with software in combination with hardware and/or firmware. For example, the subject matter described herein can be implemented in software executed by a processor or processing unit or a programmable computing machine, such as a DSP (Digital Signal Processor). The subject matter disclosed herein can be implemented in hardware form by a machine or a dedicated chip or chipset, such as an FPGA (Field-Programmable Gate Array) or an ASIC (Application-Specific Integrated Circuit). In general, the biometric information collection system 100 comprises processing electronics circuitry adapted and configured for implementing the subject matter disclosed herein.

Some embodiments of the disclosed system may be implemented, for example, using a storage medium, a computer-readable medium or an article of manufacture which may store an instruction or a set of instructions that, when executed by a machine (e.g., processor, processing circuit, or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

We claim:

1. A system for collecting biometric information comprising:
an array of sensing positions integrated into a textile and configured to collect signals associated with a subject, the array of sensing positions being distributed over an area of the textile; and
a processor in communication with the array of sensing positions, wherein the processor is configured to identify biometric information within the signals.

2. The system of claim 1, wherein the array of sensing positions comprises a plurality of discrete sensors that are each attached to the textile.

3. The system of claim 1, wherein the array of sensing positions comprises a plurality of conductive fibers integrated into the textile.

4. The system of claim 1, wherein the textile comprises a pillowcase; and
wherein the array of sensing positions is distributed over a majority of the area of the pillowcase.

5. The system of claim 1, wherein the processor is configured for continuously collecting signals from all elements in the array of sensing positions and identifying a subset of the array of sensing positions that are determined to be in contact with the subject, wherein the processor is configured to identify the biometric information within the signals by analyzing only the signals collected from the subset of the array of sensing positions.

6. The system of claim 1, comprising a data collection module connected to the array of sensing positions, wherein the data collection module is configured to transmit the signals collected by the array of sensing positions to the processor.

7. The system of claim 6, comprising an embroidered bus integrated into the textile and configured to electrically connect the array of sensing positions to the data collection module.

8. The system of claim 6, wherein one or more of the array of sensing positions is configured to operate as a reference electrode; and
wherein the data collection module is configured to provide a bias signal to the reference electrode.

9. A method for collecting biometric information, the method comprising:
positioning a textile against a portion of a body of a subject, wherein the textile comprises an array of sensing positions integrated therein;
collecting signals from the array of sensing positions;
isolating biological signals associated with the subject from the signals collected from the array of sensing positions; and
analyzing the biological signals to identify biometric information of the subject.

10. The method of claim 9, wherein collecting signals from the array of sensing positions comprises:
continuously collecting signals from all elements in the array of sensing positions; and
identifying a subset of the array of sensing positions that are determined to be in contact with the subject;
wherein isolating the biological signals associated with the subject comprises analyzing only the signals collected from the subset of the array of sensing positions.

11. The method of claim 10, comprising providing a bias signal to one or more of the array of sensing positions;
wherein identifying the subset of the array of sensing positions comprises comparing the signals collected from the array of sensing positions to the bias signal.

12. The method of claim 9, wherein isolating the biological signals associated with the subject comprises recognizing patterns in the signals that correspond to expected biometric data patterns.

13. The method of claim 12, wherein recognizing patterns in the signals comprises using an artificial intelligence or machine learning model to identify the biological signals from within the signals.

14. The method of claim 13, comprising supplying historical neurodata of the subject as training data for the artificial intelligence or machine learning model;
wherein analyzing the biological signals comprises comparing the biological signals identified from within the signals to the historical neurodata of the subject to identify the biometric information of the subject.

15. The method of claim 9, wherein isolating the biological signals associated with the subject comprises prioritizing sensing positions from among the array of sensing positions that are determined to be closest to a brain of the subject.

16. The method of claim 9, further comprising identifying additional biometric indicators within the signals collected from the array of sensing positions, the additional biometric indicators being selected from the group consisting of heart rate, heart rate variability (HRV), blood oxygen level (SpO2), temperature, and respiration rate.

17. A non-transitory computer-readable storage medium having executable instructions stored thereon, which when executed by a processing circuit of a computing device causes the computing device to:
collect signals associated with a subject from an array of sensing positions positioned against a portion of the subject's body,
isolate biological signals associated with the subject from the signals collected from the array of sensing positions; and
analyze the biological signals to identify biometric information of the subject.
